(19) United States
(12) Patent Application Publication
(10) Pub. No.: US 2010/0286539 A1
(43) Pub. Date: Nov. 11, 2010

(54) BLOOD PRESSURE MEASUREMENT DEVICE FOR PERFORMING PROCESS TAKING CHANGE OF MEASUREMENT ENVIRONMENT INTO CONSIDERATION

(75) Inventors: Shinichi Ito, Kyoto (JP); Yukiya Sawanoi, Nara (JP); Shingo Yamashita, Kyoto (JP); Mika Eto, Osaka (JP); Takahide Tanaka, Shiga (JP)

Correspondence Address: OSHA LIANG L.L.P., TWO HOUSTON CENTER, 909 FANNIN, SUITE 3500, HOUSTON, TX, 77010, US

(73) Assignee: OMRON HEALTHCARE CO., LTD., Kyoto (JP)

(21) Appl. No.: 12/810,708

(22) PCT Filed: Jan. 15, 2009
(86) PCT No.: PCT/JP2009/050458

§ 371 Date: Jun. 25, 2010

(30) Foreign Application Priority Data
Jan. 23, 2008 (JP) 2008-012923

(51) Int. Cl. A61B 5/0225

(52) U.S. Cl. 600/499

(57) ABSTRACT

A blood pressure measurement device includes a measurement fluid bag, a sensor for measuring a change in internal pressure of the measurement fluid bag, a blood pressure measurement unit for calculating a blood pressure value based on the change in internal pressure of the measurement fluid bag obtained by the sensor, an environmental information measurement unit for measuring environmental information in association with a calculation process of the blood pressure value, a storage unit for storing a measurement result, a recording processing unit for storing the calculated blood pressure value and the environmental information in the storage unit in association with each other and a notification unit for notifying a blood pressure fluctuation with respect to an environmental fluctuation based on the blood pressure value and the environmental information.

### TECHNICAL FIELD

The present invention relates to a blood pressure measurement device, and a blood pressure measurement data processing method of processing measurement data measured with the blood pressure measurement device.

### BACKGROUND ART

In a blood pressure measurement device of an oscillometric method as disclosed in Japanese Unexamined Patent Publication No. 2004-180910 (hereinafter referred to as patent document 1), a blood pressure of the measurer is measured by wrapping around and fixing to a predetermined site of a living body, namely, the measurer, an arm band (cuff) accommodating an air bladder and pressurizing or depressurizing the air bladder to measure a change in internal pressure, that is, a change from an initial state of the internal pressure.

It is important to measure and manage the blood pressure value on a daily basis for daily health management, and a home sphygmomanometer has also being widespreadly used. The blood pressure value of the human fluctuates by environmental elements such as air temperature and temperatures as well as physical elements and psychological elements, where the presence of such fluctuations is normal.

However, if the fluctuation range exceeds the limit of the human body, it becomes a risk to health of the human body. For instance, even in the house, the environmental fluctuation such as temperatures is large in winter in bathrooms, washrooms and the like, and as a result, accidents originating from blood vascular diseases are known to be likely to occur frequently from experience. A countermeasure of suppressing the environmental fluctuations themselves by heating bathrooms and the like is sometimes adopted with respect to such risks.

In the above-described sphygmomanometer, the blood pressures are recorded daily and the fluctuation of the blood pressures by elapse of time is displayed, so that data indicative of the risk of the cardiovascular system can be obtained and such data can be used by doctors for diagnosis.

Conventional sphygmomanometer may include an early morning hypertensive check function or the like for displaying and comparing the blood pressure values for respective time bands according to time information by a timer in the sphygmomanometer. However, such a function does not actually measure the environment but merely predicts and classifies ordinary actions of a person based on the time information.

Japanese Unexamined Patent Publication No. 3-231629 (hereinafter referred to as patent document 1) publicizes a thermometer that is arranged in the sphygmomanometer, so that records of blood pressure measurement results and environmental temperatures measurement results at the same time point are simultaneously displayed.

Furthermore, Japanese Unexamined Patent Publication No. 2006-280392 (hereinafter referred to as patent document 3) discloses a blood pressure measurement system for continuously measuring blood pressures using a blood pressure measurement device attached to an appropriate location of an auricle, so that an outside air temperature and a blood pressure value near the auricle are stored in association with each other, and distributions of the systolic blood pressures and the diastolic blood pressures corresponding to each other are displayed two-dimensionally.

Patent Document 1: Japanese Unexamined Patent Publication No. 2004-180910

Patent Document 2 Japanese Unexamined Patent Publication No. 3-231629

Patent Document 3 Japanese Unexamined Patent Publication No. 2006-280392

### DISCLOSURE OF THE INVENTION

### Problems to be Solved by the Invention

However, such techniques simply display by lining information on the environment, and have problems that quantitative evaluation of fluctuation of the blood pressure values by a change in the environment such as a proportion of the fluctuation of the blood pressure values cannot be easily read.

In view of such problems, it is an object of the present invention to provide a blood pressure measurement device that facilitates health management at home with respect to blood pressure fluctuations due to a change in environment, and a blood pressure measurement data processing method.

### Means for Solving the Problem

In order to achieve the above object, according to an aspect of the present invention, there is provided a blood pressure measurement device including: a measurement fluid bag; a sensor for measuring a change in internal pressure of the measurement fluid bag; a blood pressure measurement unit for calculating a blood pressure value based on the change in internal pressure of the measurement fluid bag obtained by the sensor; an environmental information measurement unit for measuring environmental information in association with a calculation process of the blood pressure value; a storage unit for storing a measurement result; a recording processing unit for storing the calculated blood pressure value and the environmental information in the storage unit in association with each other; and a notification unit for notifying a blood pressure fluctuation with respect to an environmental fluctuation based on the blood pressure value and the environmental information.

According to another aspect of the present invention, there is provided a blood pressure measurement data processing method in a blood pressure measurement device including a measurement fluid bag, a sensor for measuring a change in internal pressure of the measurement fluid bag, an environmental information measurement unit for measuring environmental information in association with a calculation process of the blood pressure value, and a storage unit for storing a measurement result; the method including the steps of: calculating a blood pressure value based on the change in internal pressure of the measurement fluid bag obtained by the sensor; measuring environmental information in association with the calculation process of the blood pressure value; storing in the storage unit the calculated blood pressure value and the environmental information in association to each other; and notifying a blood pressure fluctuation with respect to an environmental fluctuation based on the blood pressure value, the environmental information, and a comparison with a predetermined judgment standard.

### EFFECT OF THE INVENTION

According to the present invention, warnings for health management corresponding to the value of the blood pressure value itself and the proportion of the blood pressure fluctuation by the environmental fluctuation can be presented to the user. For instance, a warning on the risk in the cardiovascular system can be presented to the user if the fluctuation is greater than the proportion at which a healthy person normally fluctuates, and a warning on the possibility of abnormality in the autonomous nervous system can be presented to the user if smaller than the proportion.

### BRIEF DESCRIPTION OF THE DRAWINGS

FIG. 1 is a schematic view of an outer appearance of a sphygmomanometer according to a first embodiment.

FIG. 2 is a block diagram showing a specific example of a hardware configuration of the sphygmomanometer according to the first embodiment.

FIG. 3 is a block diagram showing a specific example of a functional configuration for performing a notifying process in the sphygmomanometer according to the first embodiment.

FIG. 4A is a view showing a display example of a stored blood pressure value and environmental temperature.

FIG. 4B is a view showing another display example of a stored blood pressure value and environmental temperature.

FIG. 5 is a flowchart for describing a process of displaying a warning or a recommended action on a display unit.

FIG. 6 is a flowchart of a blood pressure measurement process (S100 of FIG. 5) executed by a CPU in the sphygmomanometer according to the first embodiment.

FIG. 7 is a conceptual view showing a storage state of measurement data stored in a memory.

FIG. 8 is a conceptual view showing a result of classifying the blood pressure values and the environmental temperatures according to a predetermined classification standard, and storing in a classification information storage region of the memory.

FIG. 9A is a view for describing another display example displayed on the display unit.

FIG. 9B is a view for describing still another display example displayed on the display unit.

FIG. 10 is a view for describing another further display example displayed on the display unit.

FIG. 11 is a flowchart for describing a process of displaying a warning or a recommended action on a display unit in a sphygmomanometer according to a second embodiment.

FIG. 12 is a view showing a display example on the display unit when performing estimation.

FIG. 13A is a flowchart for describing a process of displaying a warning or a recommended action on a display unit in a sphygmomanometer according to a variant of the second embodiment.

FIG. 13B is a flowchart for describing a process of displaying a warning or a recommended action on a display unit in a sphygmomanometer according to another variant of the second embodiment.

### DESCRIPTION OF SYMBOLS

1 sphygmomanometer
2 main body
3 operation unit
3-1 to 3-3 button
4 display unit
5 arm band
10 air tube
13 air bladder
20 air system
21 pump
22 valve
23 pressure sensor
26, 27 drive circuit
28 amplifier
29 A/D converter
40 CPU
41 memory
50 temperature measurement unit
52 timer
101 blood pressure measurement unit
103 environmental information input unit
105 blood pressure recordation processing unit
107 environmental information recordation processing unit
109 blood pressure fluctuation calculating unit
111 display processing unit
115 informing unit

### BEST MODE FOR CARRYING OUT THE INVENTION

Embodiments of the present invention will be described below with reference to the drawings. In the following description, the same symbols are denoted for the same components and constituent elements. The names and functions of these components and constituent elements are the same.

### First Embodiment

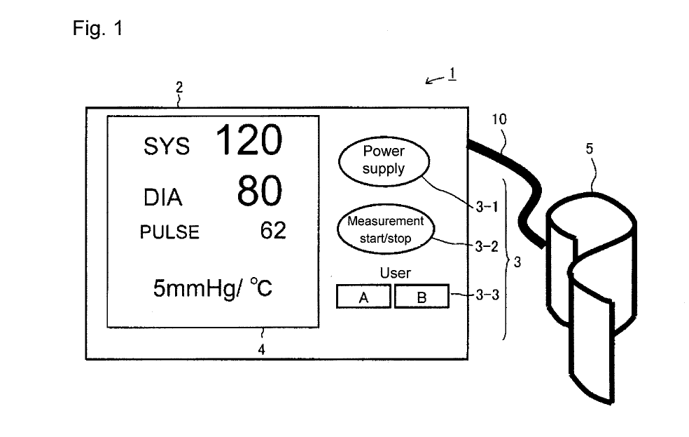

FIG. 1 is a schematic view of an outer appearance of a blood pressure measurement device (hereinafter referred to as sphygmomanometer) 1 according to a first embodiment.

With reference to FIG. 1, the sphygmomanometer 1 includes a main body 2 and an arm band 5 to be wrapped around an upper arm as a measurement site, which are connected to each other with an air tube 10. An operation unit 3 such as buttons and a display unit 4 for displaying a measurement result and the like are arranged on a front surface of the main body 2.

The operation unit 3 includes a button 3-1 for instructing ON/OFF of a power supply, a button 3-2 for instructing start/stop of measurement, and a button 3-3 for selecting a user.

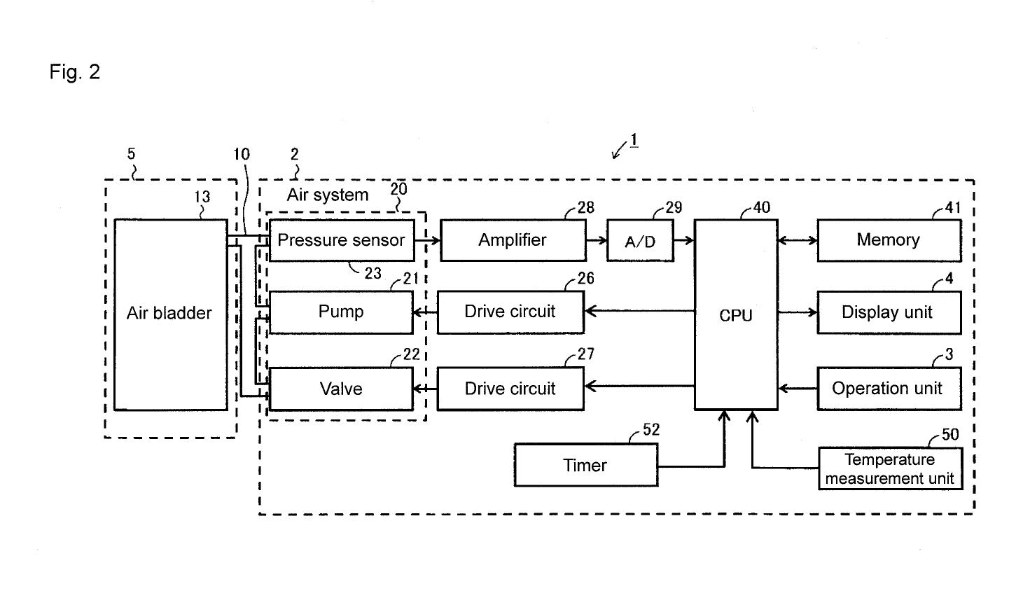

The arm band 5 includes an air bladder 13 for measurement (see FIG. 2), and the air bladder 13 is pushed against the measurement site by wrapping the arm band 5 around the upper arm as the measurement site.

FIG. 2 is a block diagram showing a specific example of a hardware configuration of the sphygmomanometer 1.

With reference to FIG. 2, the air bladder 13 is connected to an air system 20. The air system 20 includes a pressure sensor 23 for measuring a change in internal pressure of the air bladder 13, a pump 21 for supplying/exhausting air to/from the air bladder 13, and a valve 22.

The main body 2 of the sphygmomanometer 1 includes a CPU (Central Processing Unit) 40 for controlling the entire sphygmomanometer 1, an amplifier 28 connected to the air system 20, a drive circuit 26 for driving the pump 21, a drive circuit 27 for driving the valve 22, an A/D (Analog to Digital) converter 29 connected to the amplifier 28, and a memory 41 for storing programs executed by the CPU 40 and measurement results.

The CPU 40 executes a predetermined program stored in the memory 41 based on an operation signal inputted from the operation unit 3, and outputs a control signal to the drive circuit 26 and the drive circuit 27. The drive circuit 26 and the drive circuit 27 drive the pump 21 and the valve 22, respectively, according to the control signal so as to execute a blood pressure measurement operation.

The pressure sensor 23 detects the change in internal pressure of the air bladder 13, and inputs a detection signal to the amplifier 28. The inputted pressure signal is amplified to a predetermined amplitude by the amplifier 28, and is inputted to the CPU 40 after having been converted to a digital signal in the A/D converter 29. The CPU 40 executes a predetermined process based on the change in internal pressure of the air bladder 13 obtained from the pressure sensor 23, and outputs the control signal to the drive circuit 26 and the drive circuit 27 according to the result. The CPU 40 also calculates a blood pressure value based on the change in internal pressure of the air bladder 13 obtained from the pressure sensor 23, and outputs a measurement result so as to be displayed by the display unit 4.

Opening and closing of the valve 22 is controlled by the drive circuit 27 according to the control signal from the CPU 40 so as to have the air in the air bladder 13 exhausted therefrom.

Furthermore, the sphygmomanometer 1 includes a temperature measurement unit 50 for measuring an environmental temperature in time of blood pressure measurement, and a timer 52 for obtaining measurement date and time. The “environmental information” for characterizing the environment in time of blood pressure measurement includes humidity of the environment, vibration, noise, brightness, and the like in addition to the environmental temperature measured with the configuration in FIG. 2. When measuring such environmental information, a sensor for obtaining such information as a measurement value is arranged in place of the temperature measurement unit 50 or in addition to the temperature measurement unit 50, and a measurement result thereof is provided to the CPU 40.

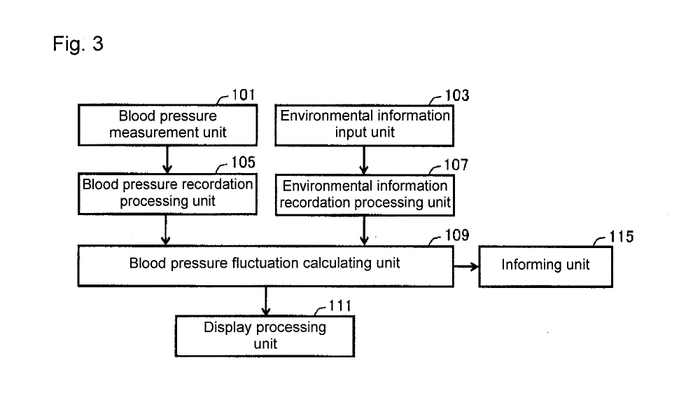

FIG. 3 is a block diagram showing a specific example of a functional configuration for performing a process of notifying (hereinafter referred to as “notifying process”) a proportion of the blood pressure fluctuation or a warning corresponding to the blood pressure fluctuation to the user. The functions shown in FIG. 3 are mainly executed by the CPU 40 when the CPU 40 executes a predetermined program stored in the memory 41. Some or all of the functions shown in FIG. 3 may be implemented by hardware.

With reference to FIG. 3, the functions for performing the notifying process in the sphygmomanometer 1 include a blood pressure measurement unit 101, an environmental information input unit 103, a blood pressure recordation processing unit 105, an environmental information recordation processing unit 107, a blood pressure fluctuation calculating unit 109, a display processing unit 111, and an informing unit 115.

The blood pressure measurement unit 101 receives the operation signal by the operation of the button 3-2, and executes the blood pressure measurement process to be described later.

The environmental information input unit 103 receives data from the sensor for obtaining the environmental information such as the temperature measurement unit 50 in cooperation with the blood pressure measurement by the blood pressure measurement unit 101.

The blood pressure recordation processing unit 105 receives the measurement result from the pressure sensor 23 through the amplifier 28 and the A/D converter 29, and performs a process of storing data in the memory 41. The environmental information recordation processing unit 107 associates the cooperating blood pressure value and the corresponding environmental information such as the environmental temperature, and performs the process of storing the data in the memory 41 with the measurement date and time obtained by the timer 52 so that the user to be measured set by the operation unit 3 can be identified.

The blood pressure fluctuation calculating unit 109 classifies the blood pressure values and the environmental temperatures stored in the memory 41 according to a predetermined classification standard, and calculates a proportion of fluctuation with respect to the environmental information data of the blood pressure. Furthermore, the display processing unit 111 performs a process of displaying on the display unit 4 the classification result or the calculation result of the blood pressure fluctuation calculating unit 109, and, as necessary, a determined warning or recommended action according to a predefined judgment standard according to the classification result or the calculation result. The notification of the warning is not limited to the display on the display unit 4, and notification may be made to the user by lighting a LED (Light Emitting Diode), ringing a buzzer, and the like by the informing unit 115.

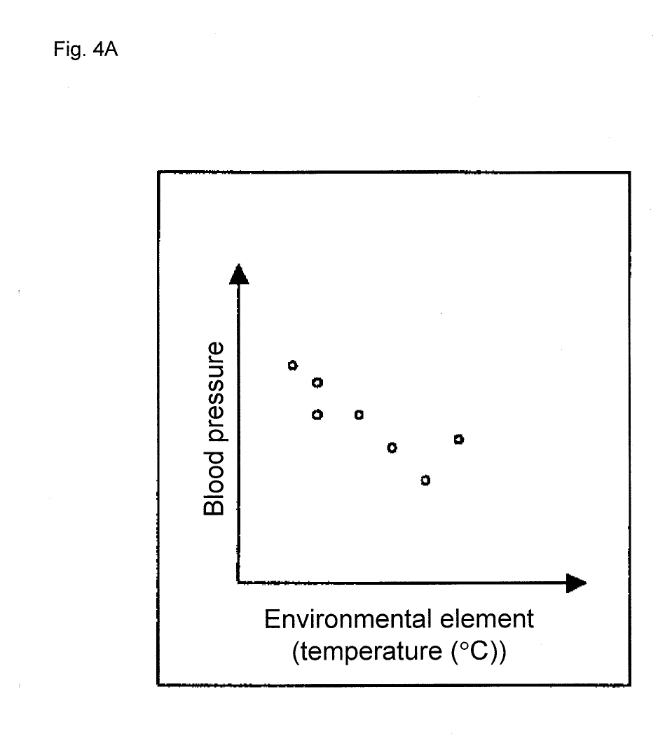

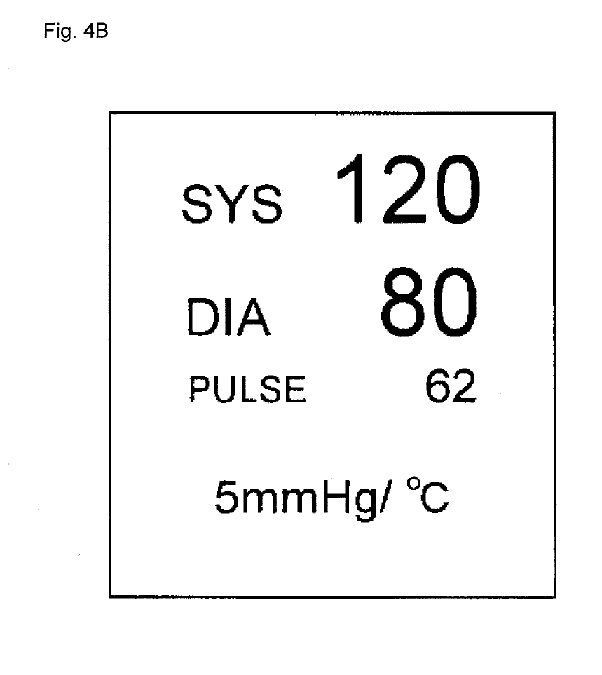

FIGS. 4A and 4B are views showing a display example of the blood pressure values and the environmental temperatures accumulated in this manner.

As shown in FIG. 4A, distribution on the environmental temperature of the blood pressure values measured at different date and time when such blood pressure values are measured can be displayed in a two-dimensional scattergram. As shown in FIG. 4B, a predetermined temperature region of interest such as a proportion of the fluctuation of the blood pressure with respect to a temperature change between 5° C. to 10° C. may be displayed as “5 mmHg/° C.”.

The state of the blood pressure fluctuation with respect to the environmental information is displayed in FIG. 4A and FIG. 4B, but also described below is the process of displaying on the display unit 4 the determined warning or recommended action according to the predefined judgment standard by the classification result or the calculation result of the blood pressure fluctuation calculating unit 109.

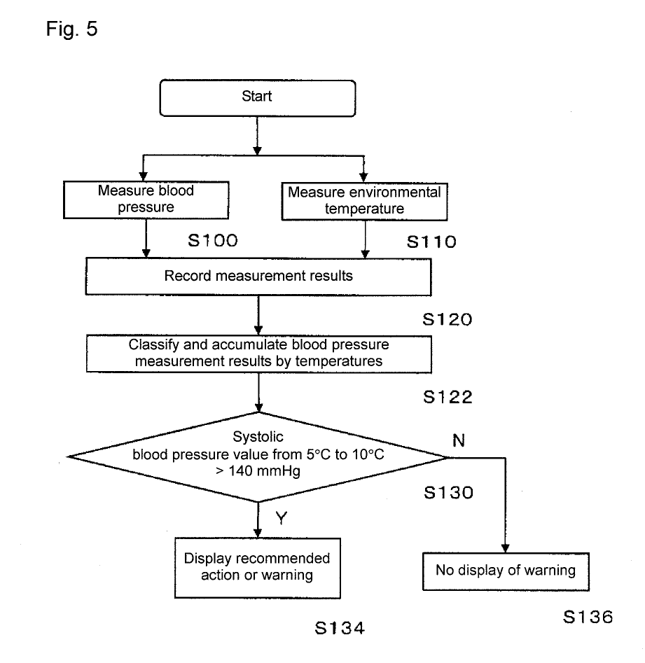

FIG. 5 is a flowchart for describing the process of displaying the warning or recommended action on the display unit 4.

With reference to FIG. 5, when the process starts, the blood pressure measurement is executed (step S (hereinafter abbreviated as “S”) 100) by the blood pressure measurement unit 101 implemented by the CPU 40, and the measurement result of the environmental temperature is received by the environmental information input unit 103 (S110).

In this case, the CPU 40 simultaneously receives the measurement date and time from the timer 52.

Subsequently, the measurement results of the blood pressure and the environmental temperature are stored in the memory 41 by the blood pressure recordation processing unit 105 and the environmental information recordation processing unit 107 implemented by the CPU 40 (S120).

The CPU 40 classifies the blood pressure values and the environmental temperatures stored in the memory 41 according to the predetermined standard, and calculates the proportion of the fluctuation with respect to the environmental information data of the blood pressure (S122). In this case, display indicating the proportion of the fluctuation with respect to the environmental temperature of the blood pressure as shown in FIG. 4B is displayed on the display unit 4.

Furthermore, the CPU 40 determines whether the systolic blood pressure value in the predetermined temperature range of interest such as between 5° C. and 10° C. is greater than a predetermined reference value such as 140 mmHg according to the classification result.

When the CPU 40 determines that the systolic blood pressure value is greater than the reference value (YES in S130), a recommended action or warning (e.g., display such as “systolic blood pressure value is rather high”), as to be described later, is displayed on the display unit 4 (S134). On the other hand, when the CPU 40 determines that the systolic blood pressure value is not greater than the reference value (NO in S130), no warning is displayed (S136).

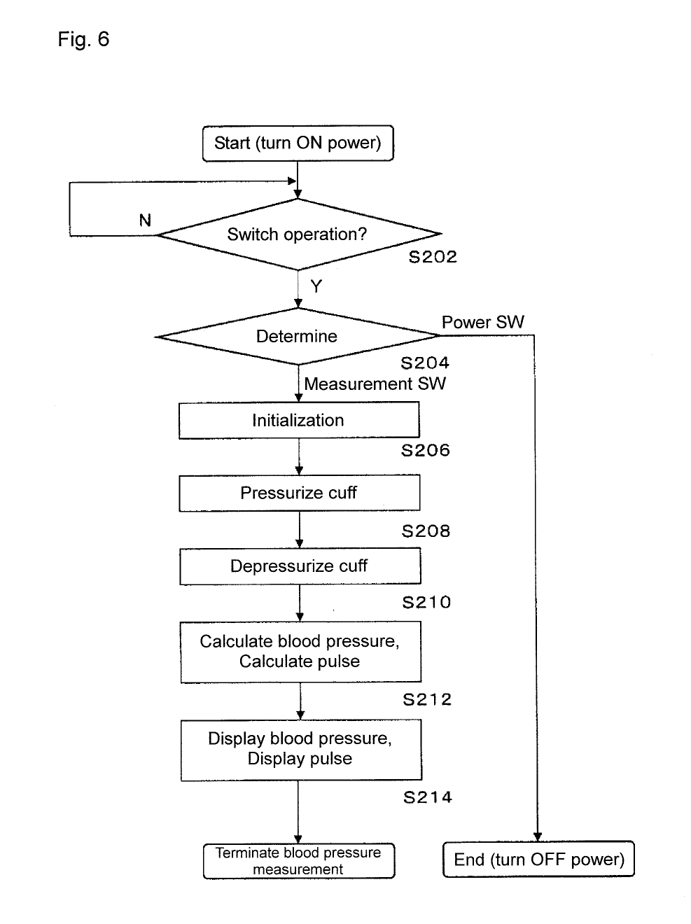

FIG. 6 is a flowchart of the blood pressure measurement process (S100 of FIG. 5) executed by the CPU 40 in the sphygmomanometer 1. The flowchart of FIG. 5 is stored in advance in the memory 41 as a program, and is read out and executed by the CPU 40. The process shown in FIG. 6 starts when the power is supplied to the CPU 40 after the power supply switch 3-1 is operated, for example.

With reference to FIG. 6, the CPU 40 first determines presence of the switch operation (step S202). The CPU 40 waits until the switch operation is detected (NO in S202). When the switch operation is detected (YES in S202), the type of the operated switch is determined (S204).

The process proceeds to S206 if determined that the measurement switch 3-2 is operated in S204, and the power is turned OFF and the process is terminated if determined that the power switch 3-1 is operated.

The process related to the blood pressure measurement shown in S206 to S214 will be described first. The CPU 40 first controls each unit and exhausts the air in the air bladder 13 to perform 0 mmHg correction of the pressure sensor 23 as the initialization process of the sphygmomanometer 1 (step S206). The CPU 50 then controls each unit and pressurizes up to about the level of the systolic blood pressure of the subject+40 mmHg (step S208). The cuff pressure is then gradually depressurized (step S210). In the depressurization process, the cuff pressure is detected with the pressure sensor 23, and the CPU 40 calculates the blood pressure (systolic blood pressure and diastolic blood pressure) values as well as the number of pulses based on the detected pressure (step S212). The calculated blood pressure values and the number of pulses are displayed on the display unit 4 (S214). The processes for the blood pressure measurement in S208 to S212 are similar to that of the conventional electronic sphygmomanometer. In the present case, the blood pressure measurement is performed in the depressurization process, but may be performed in the pressurization process.

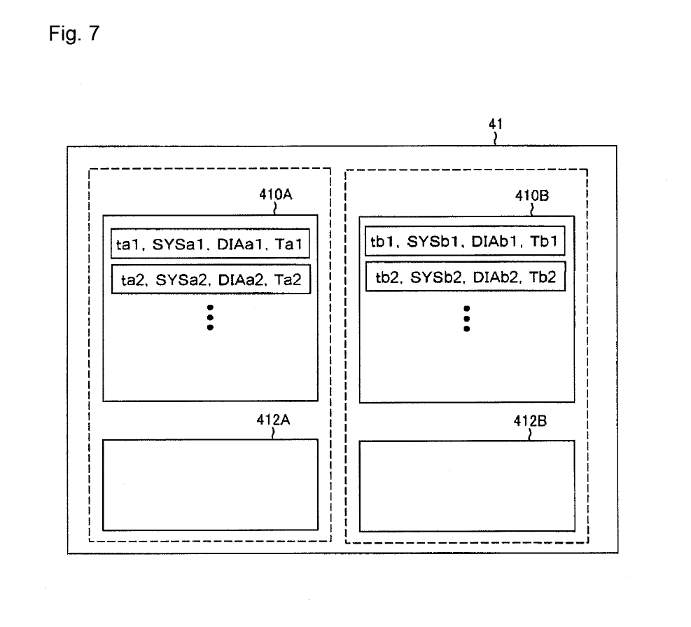

FIG. 7 is a conceptual view showing a storage state of the measurement data stored in the memory 41.

In the example shown in FIG. 7, the data storage region is first separated according to whether the measurement result is on the user A or on the user B.

For instance, a systolic blood pressure value SYSa1, a diastolic blood pressure value DIAa1, and an environmental temperature Ta1 at date and time ta1 with respect to the user A are associated to each other and stored in the measurement data storage region 410A. The measurement results at a different date and time such as date and time ta2 with respect to the user A are similarly stored in the measurement data storage region 410A.

Similarly, a systolic blood pressure value SYSb1, a diastolic blood pressure value DIAb1, and an environmental temperature Tb1 at date and time tb1 with respect to the user B are associated to each other and stored in the measurement data storage region 410B. The measurement results at a different date and time such as date and time tb2 with respect to the user B are similarly stored in the measurement data storage region 410B.

The classifying information storage region 412A stores the result of classifying the blood pressure measurement values for the user A according to the predetermined classification standard, and the classifying information storage region 412B stores the result of classifying the blood pressure measurement values for the user B according to the predetermined classification standard.

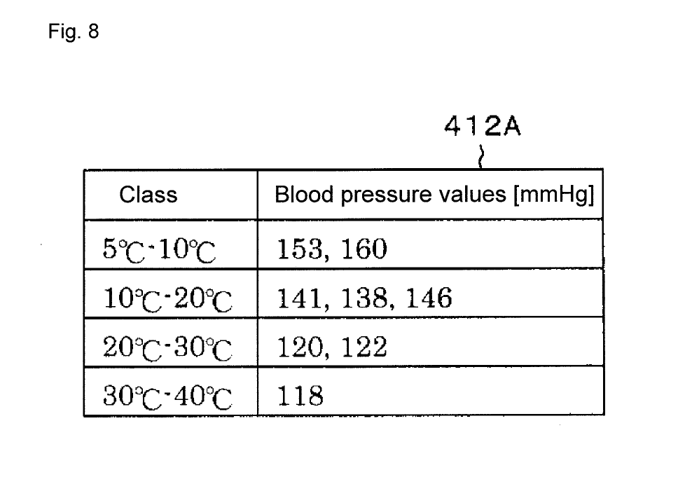

FIG. 8 is a conceptual view showing a result of classifying the blood pressure values and the environmental temperatures according to the predetermined classification standard and storing in the classification information storage region 412A of the memory 42 in S122 of FIG. 5.

As shown in FIG. 8, the environmental temperatures is classified into regions from 5° C. to 10° C., from 10° C. to 20° C., from 20° C. to 30° C., and from 30° C. to 40° C., and the measured blood pressure values are respectively classified to the corresponding regions.

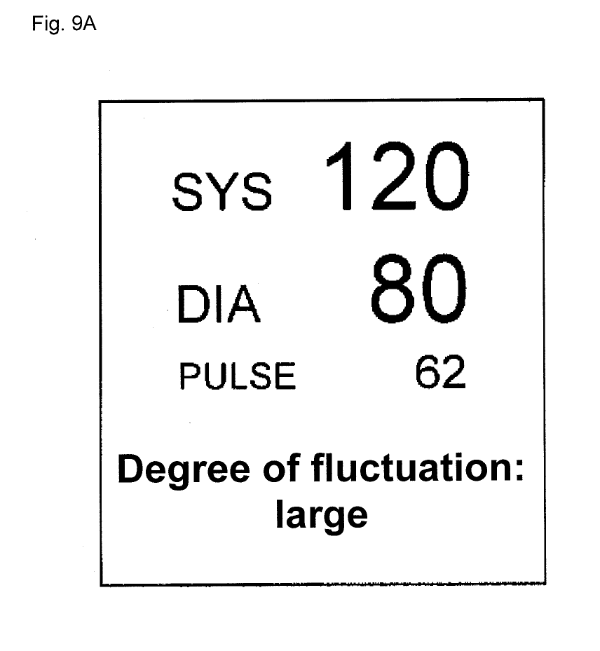

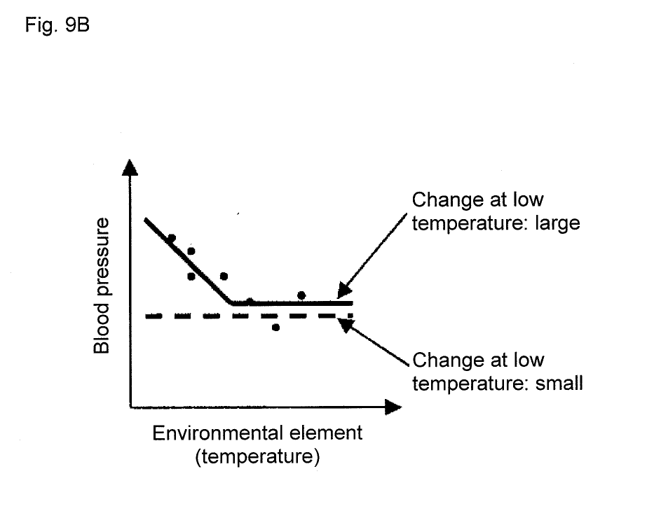

FIG. 9A and FIG. 9B are views for describing another display example displayed on the display unit 4 in S134 of FIG. 5.

As shown in FIG. 9B, when the change in blood pressure in a low temperature range of the environmental temperature is small, a graph indicates substantially horizontal line as shown with a broken line. On the other hand, when the proportion of the blood pressure fluctuation is greater than a predetermined reference value in a low temperature range of the environmental temperature such as in the range from 5° C. to 10° C., the display “degree of fluctuation: large” is made as in FIG. 9A. Instead of simply displaying “when proportion of blood pressure fluctuation is greater than predetermined reference value”, the degree of fluctuation may be divided into ranks and the type thereof may be displayed.

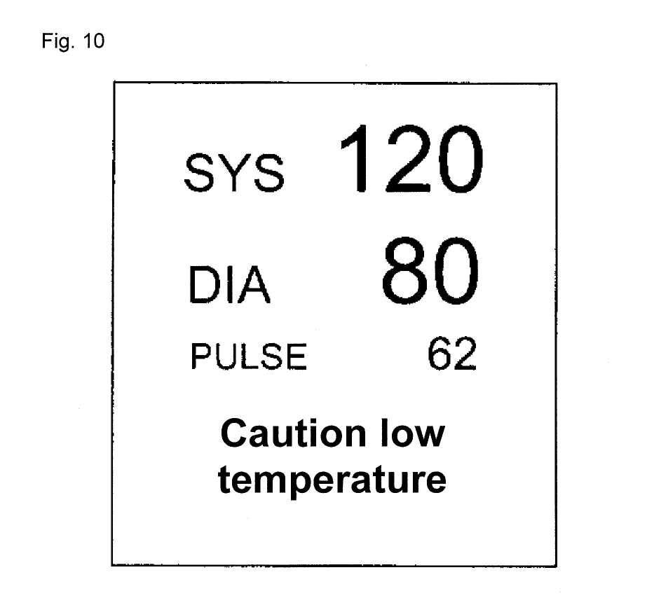

FIG. 10 is a view for describing another further display example displayed on the display unit 4 in S134 of FIG. 5.

In FIG. 9A and FIG. 9B, the warning on the blood pressure fluctuation is displayed, but in FIG. 10, the recommended action is displayed. For example, if the proportion of the blood pressure fluctuation is greater than the predetermined reference value in the low temperature range of the environmental temperature such as in the range from 5° C. to 10° C., the display “caution low temperature” is made to recommend the user to avoid sudden transition to the “low temperature environment” (e.g., taking a bath in a bathroom not sufficiently warmed). The display of the recommended action may be other than the example of FIG. 10 and may alternatively be “take bath after warming the bathroom”.

With the above-described configuration, the value of the blood pressure value itself or the warning for health management corresponding to the proportion of the blood pressure fluctuation by the fluctuation of the environment may be presented to the user. If greater than the proportion at which a healthy person normally fluctuates, the risk in the cardiovascular system can be presented to the user, and if smaller than the proportion, the warning on the possibility of abnormality in the autonomous nervous system can be presented to the user.

### Second Embodiment

In a second embodiment, the operation of a sphygmomanometer 1 capable of displaying a warning or presenting a recommended action to the user by estimating a blood pressure value even in a temperature region in which the blood pressure measurement value may not necessarily exist will be described. The hardware configuration of the sphygmomanometer of the second embodiment is basically similar to the configuration of the sphygmomanometer of the first embodiment, and differs in the calculation process with respect to the blood pressure fluctuation by the CPU 40 as to be described below.

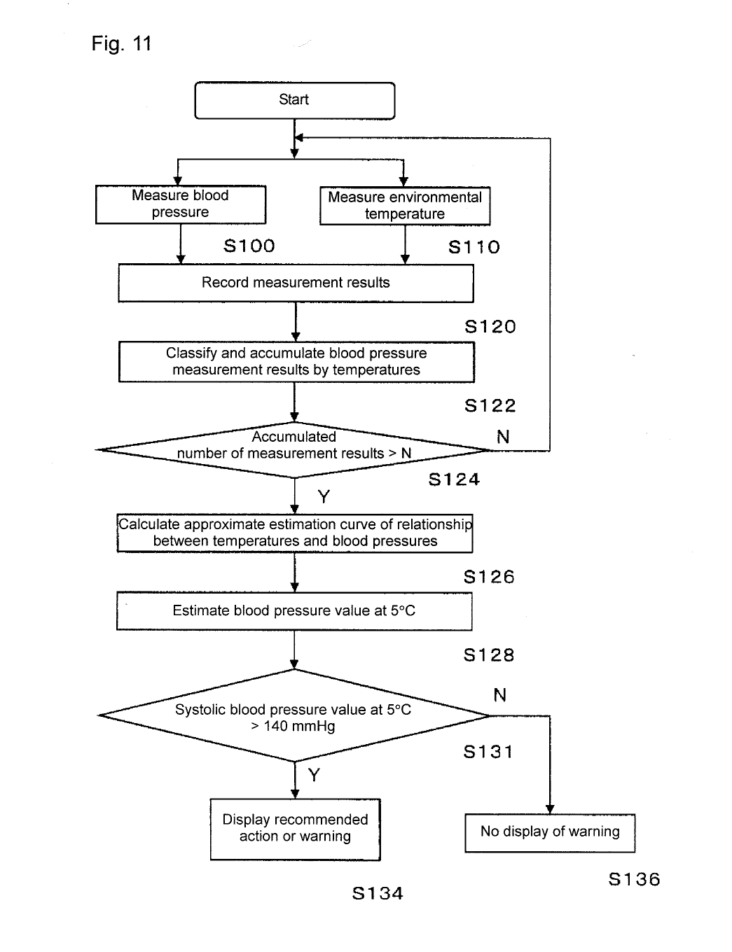

FIG. 11 is a flowchart for describing a process of displaying a warning or recommended action on the display unit 4 in the sphygmomanometer of the second embodiment.

With reference to FIG. 11, when the process starts, the blood pressure measurement is first executed (step S100) by the blood pressure measurement unit 101 implemented by the CPU 40, and the measurement result of the environmental temperature is received by the environmental information input unit 103 (step S110).

In this case, the CPU 40 simultaneously receives the measurement date and time from the timer 52.

Subsequently, the measurement results of the blood pressure and the environmental temperature are stored in the memory 41 (S120) by the blood pressure recordation processing unit 105 and the environmental information recordation processing unit 107 implemented by the CPU 40.

The CPU 40 classifies the blood pressure values and the environmental temperatures stored in the memory 41 according to a predetermined classification standard, and calculates a proportion of the fluctuation of the blood pressure with respect to the environmental information data (S122). In this case, the display showing the proportion of the fluctuation of the blood pressure with respect to the environmental temperature as shown in FIG. 4 is displayed on the display unit 4.

If the accumulated number of the measurement results for the same user is less than a predetermined number N (S124), the process returns to steps S100 and S110, and if the accumulated number of the measurement results is greater than the predetermined number N (S124), the CPU 40 calculates an approximate estimation curve of the relationship between the temperatures and blood pressures (S126). In place of a line, a polynomial of a predetermined order or the like may be used for the approximate estimation curve.

The CPU 40 estimates a blood pressure value at 5° C., for example, which does not exist as a measurement value, from the approximate estimation curve obtained above (S128).

The CPU 40 determines whether or not the estimated value of the systolic blood pressure value at 5° C. is greater than a predetermined reference value such as 140 mmHg according to the estimation result.

When the CPU 40 determines that the systolic blood pressure value is greater than the reference value (YES in S131), the recommended action or warning (e.g., display such as “systolic blood pressure value is rather high”) as described above is made on the display unit 4 (S134). On the other hand, when the CPU 40 determines that the estimation value of the systolic blood pressure is not greater than the reference value (NO in S131), no warning is displayed (S136).

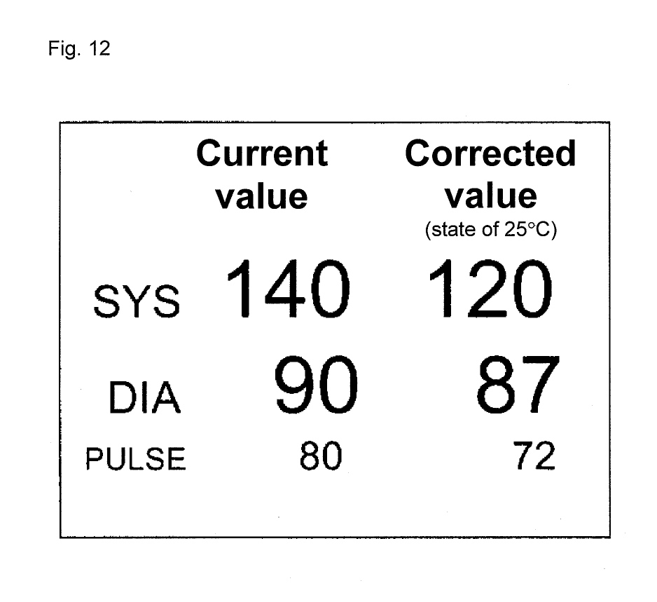

FIG. 12 is a view showing a display example on the display unit 4 when performing such an estimation.

A value converted to a temperature region without a measurement point is displayed as a “corrected value” with respect to the current measurement value. For instance, the blood pressure value at 5° C. is estimated based on the measurement results at the range from 10° C. to 30° C. In some cases, the measurement result at 10° C. may be displayed as “current location”, and the converted value in the standard state (e.g., 25° C.) may be displayed.

The temporal change of the risk under a low temperature environment can be checked by calculating the estimated value (or corrected value) even in a case where the measurement value in such a low temperature environment is not yet actually obtained. Alternatively, the temporal change can be more accurately checked with the value estimated to be measured under a constant condition if the measurement value is re-converted to that in the standard state.

### Variant of Second Embodiment

In the second embodiment, the display of the warning or recommended action is made at the time when the user measures the blood pressure, but the sphygmomanometer 1 may constantly monitor whether or not the environmental temperature is in a dangerous region, so as to display a warning or notify the warning (warning sound or the like) when in the dangerous region.

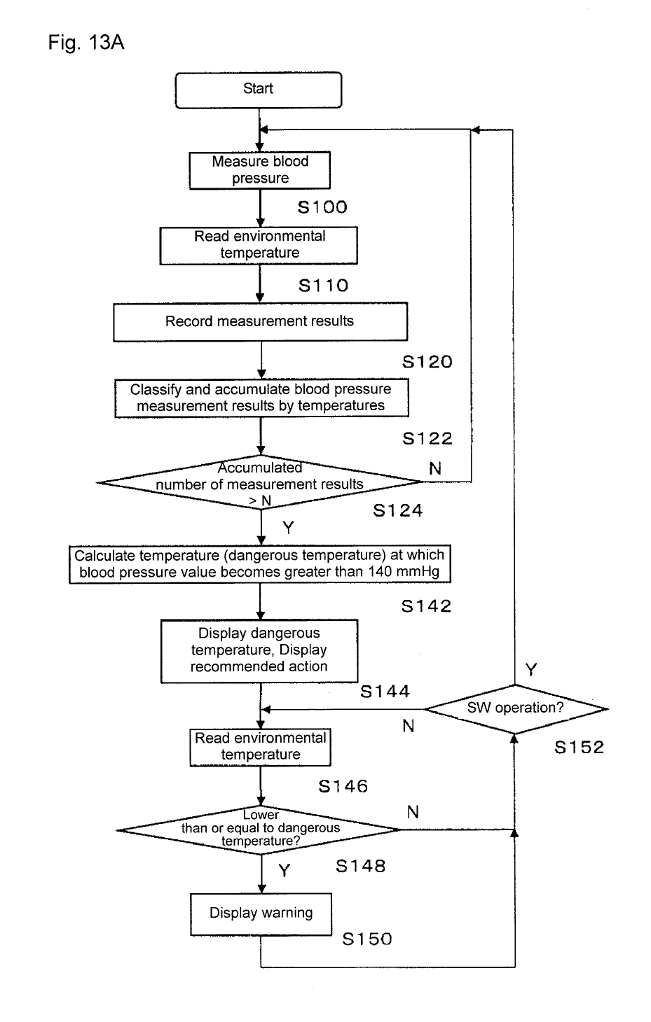

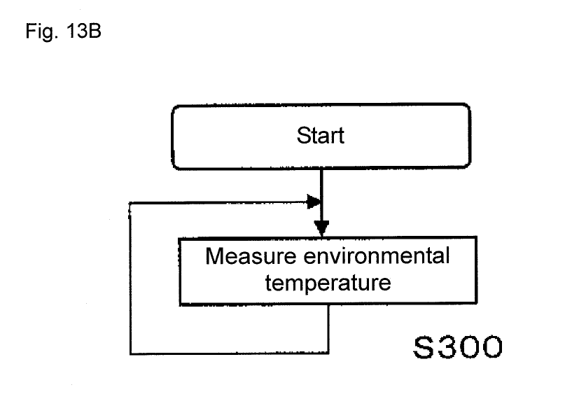

FIG. 13A and FIG. 13B are flowcharts for describing a process of displaying a warning or recommended action on the display unit 4 in a sphygmomanometer of a variant of the second embodiment.

In FIG. 13A, the steps denoted with the same symbols from steps S100 to S124 are similar to those of FIG. 11. With reference to FIG. 13A, when the process starts, blood pressure measurement is first executed (step S100) by the blood pressure measurement unit 101 implemented by the CPU 40. On the other hand, in FIG. 13B, the processing routine for the environmental temperature measurement is constantly operating, and the environmental temperature is measured (step S300) by the temperature measurement unit 50.

Following the measurement of the blood pressure, the environmental information recordation processing unit 107 reads the measured environmental temperature from the temperature measurement unit 50 (S110′), and the CPU 40 simultaneously receives the measurement date and time from the timer 52.

Subsequently, the measurement results of the blood pressure and the environmental temperature are stored in the memory 41 (S120) by the blood pressure recordation processing unit 105 and the environmental information recordation processing unit 107 implemented by the CPU 40.

The CPU 40 classifies the blood pressure values and the environmental temperatures stored in the memory 41 according to a predetermined classification standard, and calculates a proportion of the fluctuation of the blood pressure with respect to the environmental information data (S122). In this case, the display showing the proportion of the fluctuation of the blood pressure with respect to the environmental temperature as shown in FIG. 4B is displayed on the display unit 4.

If the accumulated number of measurement results for the same user is greater than a predetermined number N (YES in S124), the CPU 40 calculates an approximate estimation curve of the relationship between the blood pressure and the temperature to calculate an anticipated temperature (dangerous temperature) at which the blood pressure becomes greater than the predetermined value such as 140 mmHg (S142). Other than a line, a polynomial of a predetermined order or the like may be used for the approximate estimation curve.

The display of the calculated dangerous temperature and the recommended action (“caution required to move to environment lower than or equal to XX° C. (dangerous temperature)” or the like) is made (S144).

Subsequently, the environmental information recordation processing unit 107 implemented by the CPU 140 receives the environmental temperature from the temperature measurement unit 50 (S146). If lower than or equal to the dangerous temperature (YES in S148), the warning (“environmental temperature is “dangerous temperature” or the like) is displayed, and whether or not the switch operation is performed is determined (S152). If the switch operation is made, the process returns to steps S100 and S110. If the switch operation is not made, the process returns to step S146.

If not lower than or equal to the dangerous temperature (NO in S148), whether or not the switch operation is made is determined (S152). If the switch operation is made, the process returns to steps S100 and S110. If the switch operation is not made, the process returns to step S146.

Therefore, the health management at home with respect to the blood pressure fluctuation due to a change in environment can be facilitated as the environmental temperature is constantly monitored by the sphygmomanometer.

The embodiments disclosed herein are illustrative in all aspects and should not be construed as being restrictive. The scope of the present invention is defined by the Claims rather than by the description provided above, and meanings equivalent to the Claims and all modifications within the scope are intended to be encompassed herein.

1. A blood pressure measurement device comprising:
a measurement fluid bag;
a sensor for measuring a change in internal pressure of the measurement fluid bag;
a blood pressure measurement unit for calculating a blood pressure value based on the change in internal pressure of the measurement fluid bag obtained by the sensor;
an environmental information measurement unit for measuring environmental information in association with a calculation process of the blood pressure value;
a storage unit for storing a measurement result;
a recording processing unit for storing the calculated blood pressure value and the environmental information in the storage unit in association with each other; and
a notification unit for notifying a blood pressure fluctuation with respect to an environmental fluctuation based on the blood pressure value and the environmental information.

2. The blood pressure measurement device according to claim 1, further comprising:
a display unit, wherein
the notification is made by displaying on the display unit a numerical value indicating a proportion of the blood pressure fluctuation with respect to the environmental fluctuation.

3. The blood pressure measurement device according to claim 1, further comprising:
a display unit, wherein
the notification is made by displaying on the display unit information on a range of an environment having the blood pressure fluctuation possibly outside a predetermined allowable range.

4. The blood pressure measurement device according to claim 1, further comprising:
a classifying unit for classifying the blood pressure value and the environmental information into a predetermined region of the environmental information, wherein
the notification indicates that the blood pressure value in a region of interest of the environmental information is included in a predetermined region.

5. The blood pressure measurement device according to claim 1, further comprising:
a display unit, wherein
the notification is made by displaying on the display unit that a proportion of the blood pressure fluctuation with respect to the environmental fluctuation is classified into a predetermined rank.

6. The blood pressure measurement device according to claim 2, further comprising a recommended notification unit for notifying to urge a recommended action to a user.

7. The blood pressure measurement device according to claim 1, further comprising:
an approximation unit for calculating an approximate estimation curve of the blood pressure value with respect to the environmental information based on the measured environmental information and the calculated blood pressure value; and
an estimation unit for calculating a blood pressure value at a non-measured value of environmental information based on the approximate estimation curve.

8. The blood pressure measurement device according to claim 1, further comprising a monitoring region calculating unit for calculating a monitoring range of the environmental information where the blood pressure value is included in a predetermined region based on the information on the blood pressure fluctuation with respect to the environmental fluctuation.

9. The blood pressure measurement device according to claim 8, further comprising:
a display unit; and
a monitoring unit for displaying a warning on the display unit according to a detection that the environmental information is in the monitoring range.

10. A blood pressure measurement data processing method in a blood pressure measurement device including a measurement fluid bag, a sensor for measuring a change in internal pressure of the measurement fluid bag, an environmental information measurement unit for measuring environmental information in association with a calculation process of the blood pressure value, and a storage unit for storing a measurement result; the method comprising the steps of:
calculating a blood pressure value based on the change in internal pressure of the measurement fluid bag obtained by the sensor;
measuring environmental information in association with the calculation process of the blood pressure value;
storing in the storage unit the calculated blood pressure value and the environmental information in association to each other; and
notifying a blood pressure fluctuation with respect to an environmental fluctuation based on the blood pressure value, the environmental information, and a comparison with a predetermined judgment standard.

11. The blood pressure measurement device according to claim 3, further comprising a recommended notification unit for notifying to urge a recommended action to a user.

12. The blood pressure measurement device according to claim 4, further comprising a recommended notification unit for notifying to urge a recommended action to a user.

13. The blood pressure measurement device according to claim 5, further comprising a recommended notification unit for notifying to urge a recommended action to a user.
